(19) United States
(12) Patent Application Publication
(10) Pub. No.: US 2014/0045930 A1
(43) Pub. Date: Feb. 13, 2014

(54) ASSAY FOR ARGINYL HYDROXYLASE ACTIVITY

(71) Applicants: Christopher Joseph Schofield, Oxford (GB); Chia-Hua Ho, Oxford (GB); Alexander Wolf, Oxford (GB); Wei Ge, Oxford (GB)

(72) Inventors: Christopher Joseph Schofield, Oxford (GB); Chia-Hua Ho, Oxford (GB); Alexander Wolf, Oxford (GB); Wei Ge, Oxford (GB)

(21) Appl. No.: 13/982,639

(22) PCT Filed: Jan. 30, 2012
(86) PCT No.: PCT/GB12/50191

§ 371 Date: Oct. 15, 2013

(30) Foreign Application Priority Data
Jan. 31, 2011 (GB) 1101664.9

(51) Int. Cl. C12Q 1/26; C12P 21/00; A61K 45/00

(52) U.S. Cl. CPC C12Q 1/26; A61K 45/00; C12P 21/00
USPC 514/456; 435/25; 514/563; 514/561; 514/557; 435/375; 435/68.1

(57) ABSTRACT

The present invention relates to assays for monitoring activity of YcfD activity, in particular, to assays for identifying modulators of YcfD activity. The present invention also relates to the use of YcfD inhibitors as antibiotics. The invention also relates to methods for introducing hydroxyarginine residues into proteins.

### FIELD OF THE INVENTION

The present invention relates to assays for monitoring activity of a newly identified family of ribosome hydroxylases, in particular, to assays for identifying modulators of ribosome hydroxylase activity. The present invention also relates to the use of such modulators, and in particular inhibitors as antibiotics. The invention also relates to methods for introducing hydroxyarginine residues into proteins.

### BACKGROUND TO THE INVENTION

In animals the hydroxylation of intracellularly located proteins, including N-methyl lysine demethylation via hydroxylation, is more common than once perceived. Hydroxylation of collagen at the 3- and 4-positions of prolyl-residues and at the 5-position of lysine residues has long been established. More recently, the hypoxia inducible transcription factor (HIF) has been shown to undergo prolyl 4-hydroxylation (catalyzed by PHD/EGLN enzymes) and asparaginyl 3-hydroxylation; these modifications are of central importance in the oxygen dependent regulation of the hypoxic response in animals. The HIF asparaginyl hydroxylase, factor inhibiting HIF (FIH) also catalyses asparaginyl hydroxylation of multiple ankyrin repeat domain protein substrates. Following the identification of the roles of hydroxylation in the hypoxic response, related enzymes have been shown to catalyze lysyl 5-hydroxylation of splicing-related proteins (i.e. by JMJD6) and the demethylation of N-8-methyl lysine histone residues. All of the aforementioned reactions are catalyzed by Fe(II) and 2-oxoglutarate (2OG) oxygenases which are a large superfamily with likely >60 human members. These enzymes couple the two-electron oxidation of their ‘prime’ substrate to the oxidative decarboxylation of 2OG to give carbon dioxide (CO₂) and succinate.

The evidence that 2OG oxygenase catalysed post-translational hydroxylation is common in animals and likely other eukaryotes, raises the question as to whether it occurs in prokaryotes.

### SUMMARY OF THE INVENTION

We describe work demonstrating that the Escherichia coli protein YcfD is a 2-oxoglutarate (2OG) oxygenase, and in particular show that YcfD is an argininyl hydroxylase. The inventors also identify a substrate for this 2OG oxygenase, namely the E. coli ribosomal protein, L16.

Accordingly, the present invention provides a method for assaying YcfD activity, the method comprising contacting a peptide containing one or more arginine residues, with a YcfD polypeptide and determining whether an arginine residue in said peptide is hydroxylated.

The invention also provides a method for identifying an inhibitor of YcfD oxygenase activity, the method comprising contacting a YcfD polypeptide and an arginine containing peptide with a test agent under conditions suitable for oxygensae activity, and monitoring for hydroxylation of the arginine of said peptide.

The invention also provides a method for identifying a modulator of protein translation, the method comprising contacting a cell which expresses YcfD with a test agent and determining whether the test agent modulates the YcfD regulation of protein translation.

The invention further provides an inhibitor or activator of 2-OG oxygenase activity for use in modulating argininyl hydroxylation by YcfD of a ribosomal protein or a fragment or variant thereof comprising an arginine residue, or for use in modulating protein translation.

The invention further provides a method for introducing hydroxyarginine into a protein by employing a YcfD polypeptide to catalyse such a modification.

### BRIEF DESCRIPTION OF THE FIGURES

FIG. 1:

### Panel A

[¹⁴C] 2OG decarboxylation assay. 2OG turnover catalysed by YcfD without a prime substrate. Conversion of 2OG was calculated from the percentage of 1-[¹⁴C]-2OG that had been converted to ¹⁴CO₂ gas. Assays were performed in triplicate; error bars give standard deviations.

### Panel B

MALDI-time-of-flight (MALDI-TOF) mass spectrometric analysis of peptide RLLPAVSEATIRRL (SEQ ID NO: 10). Incubation of peptide RLLPAVSEATIRRL (20 μM) with ascorbate (2 mM), Fe(II) (200 μM), 2OG (200 μM) and YcfD (2 μM) (II). (I) is control without YcfD. (III) includes all components except 2OG, (IV) includes all components except Fe(II).

### Panel C

Hydroxylation activity of truncated variants of RLLPAVSEATIRRL.

FIG. 2:

### Panel A

Anti-GFP immunoprecipitation assay after overexpression of either GFP or GFP-YcfD in BL21(DE3) E. coli. Coomassie staining of a 1D SDS Page. Indicated bands were excised and analysed by LC-MS/MS after tryptic digest. *=GFP; **=GFP-YcfD; ***=ribosomal protein L16.

### Panel B

MS fragmentation (MS/MS) analysis of endogenous ribosomal protein L16 reveals hydroxylation of arginine 81 (R-81). L16 was co-purified after GFP-YcfD overexpression and anti-GFP pulldown. Insert shows the MH³⁺ peptide precursor ion that was fragmented, the b and y fragment ions are indicated.

FIG. 3:

Alignment of different prokaryotic YcfD homologues: Escherichia coli (Ec; NP_{—}415646), Shigella boydii (Sb; CP001063), Salmonella enterica (Se; AM933173), Klebsiella pneumoniae (Kp; CP000647), Erwinia pyrifoliae (Ep; FN392235). Conserved amino acids are highlighted with the dark backgrounds. Semi-conserved amino acids are highlighted with the light backgrounds. The alignment was performed using ClustalW and visualized using jalview software.

### BRIEF DESCRIPTION OF THE SEQUENCES OF THE INFORMAL SEQUENCE LISTING

SEQ ID NO: 1 is the amino acid sequence of E. coli YcfD.

SEQ ID NO: 2 is the amino acid sequence of the JmjC domain of YcfD (residues 92 to 219 of SEQ ID NO: 1).

SEQ ID NO: 3 is the amino acid sequence of E. coli L16.

SEQ ID NOs: 4 to 7 are the amino acids sequences of YcfD homologues in Shigella boydii (Sb; CP001063), Salmonella enterica (Se; AM933173), Klebsiella pneumoniae (Kp; CP000647), Erwinia pyrifoliae.

SEQ ID NOs: 8 and 9 are the nucleotide sequences of the primers used to verify inactivation of YcfD.

SEQ ID NOs: 10 to 12 are the amino acid sequences of the synthetic peptides used to assess hydroxylation by YcfD.

SEQ ID NOs: 13 to 27 are the amino acid sequences of the synthetic peptides shown in Table 2.

### DETAILED DESCRIPTION OF THE INVENTION

The present inventors have shown that YcfD has 2-oxoglutarate (2-OG) dependent oxygenase activity and in particular catalyses hydroxylation of argininyl residues. A substrate for this argininyl hydroxylase activity has been identified, namely the E. coli ribosomal protein L16.

The present invention provides a method for assaying YcfD activity, the method comprising contacting a YcfD polypeptide with a peptide containing an arginine residue, and determining whether the peptide is hydroxylated at the arginine residue.

A YcfD polypeptide in accordance with the present invention is typically YcfD from E. coli or a homologue thereof, a variant thereof which retains argininyl hydroxylase activity, or a fragment of any thereof which retains argininyl hydroxylase activity. The sequence of YcfD from E. coli is set out in SEQ ID NO: 1. Homologues thereof may be from other bacterial species including for example Shigella boydii, Salmonella enterica, Klebsiella pneumoniae and Erwinia pyrifoliae. Homologues thereof are set out in SEQ ID NOs: 4 to 7.

The YcfD polypeptide may comprise the sequence shown in SEQ ID NO: 1, or may be a fragment or variant of SEQ ID NO: 1 having argininyl hydroxylase activity. Fragments of YcfD are described in more detail below. The YcfD polypeptide may have an amino acid sequence having at least about 60% sequence identity, for example at least about 70% sequence identity, with SEQ ID NO: 1 over its entire length or over an active fragment thereof (such as SEQ ID NO: 2), typically greater than about 80% or 90%, such as about 95% or about 99% sequence identity.

Sequence identity may be calculated using any suitable algorithm. For example, the UWGCG Package provides the BESTFIT program can be used to infer homology (for example used on its default settings) (Devereux et al. (1984) Nucleic Acids Research 12, p 387-395). The PILEUP and BLAST algorithms can be used to infer homology or line up sequences (typically on their default settings), for example as described in Latched (1993) J. Mol. Evol. 36:290-300; Latched et al. (1990) J. Mol. Biol. 215:403-10.

The YcfD polypeptide may be a polypeptide encoded by any naturally occurring YcfD gene. The naturally occurring YcfD gene may encode the sequence shown in SEQ ID NO: 1 or may encode a variant. Such variants may include allelic variants and the deletion, modification or addition of single amino acids or groups of amino acids within the protein sequence, as long as the polypeptide retains argininyl hydroxylase activity.

Amino acid substitutions of SEQ ID NO: 1, or of a fragment thereof may be made, for example from about 1, 2 or 3 to about 10, 20 or 30 substitutions.

Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Variant polypeptides within the scope of the invention may be generated by any suitable method, for example by point mutation or gene shuffling techniques.

The present invention also includes use of active portions, fragments, derivatives and functional mimetic of the polypeptides of the invention. An “active portion” of a polypeptide means a peptide which is less than said full-length polypeptide, but which retains argininyl hydroxylase activity. An active fragment of YcfD may typically be identified by monitoring for 2-OG oxygenase activity as described in more detail below. Such an active fragment may be included as part of a fusion protein.

The fragment may have up to about 60, 70, 80, 100, 150, 200, 300 or 350 amino acids. The fragment may comprise any region from the amino acid sequence shown in SEQ ID NO: 1, such as from amino acid 2, 3, 4, 5 or about 10 to about amino acid 330, 340, 350, 360 or 370. Useful fragments include N-terminal (or C-terminal) truncated fragments i.e., fragments comprising an N-terminal deletion, such as fragments comprising residues 10 to 373, 20 to 373 or 25 to 373 of the amino acid sequence shown in SEQ ID NO: 1. Useful fragments also include fragments comprising C-terminal truncations such as fragments comprising residues 1 to 370, 1 to 360 or 1 to 340 of the amino acid sequence shown in SEQ ID NO: 1. Useful fragments also include fragments comprising both N-terminal and C-terminal truncations, such as fragment comprising residues 10 to 370, 20 to 360 or 25 to 340 of the amino acid sequence shown in SEQ ID NO: 1. Examples of a specific truncated fragment that may be used in the invention is shown in SEQ ID NO: 2 (residues 92 to 219). Residues 92 to 219 comprise the JmjC domain of YcfD. Other suitable fragments may readily be identified, for example by comparing the YcfD amino acid sequence to the amino acid sequence of one or more known 2-OG dependent oxygenase and identifying which regions are homologous to regions having catalytic activity. The regions having catalytic activity are typically included in the active fragments. Such fragments can be used to construct chimeric molecules.

Fragments of any YcfD polypeptide having at least about 60%, such as at least about 70%, 80%, 90%, 95% or 99% sequence identity to the amino acid sequence shown in SEQ ID NO: 1, which fragments have argininyl hydroxylase activity may also be used in an assay of the invention and are encompassed within the term “YcfD polypeptide” used herein.

The YcfD polypeptide may comprise one or more particular site directed mutations.

The YcfD polypeptides may be synthetically prepared. The polypeptides may be chemically or biochemically modified, e.g. post-translationally modified. For example, they may be glycosylated or comprise modified amino acid residues. They may also be modified by the addition of histidine residues (typically six), or other sequence tags such as a maltose binding protein tag or integrin tag, to assist their purification or by the addition of a nuclear localisation sequence to promote translocation to the nucleus or mitochondria, and or by post translational modification including hydroxylation or phosphorylation. Polypeptides of the invention may be GST or other suitable fusion polypeptides. The YcfD polypeptide may also be modified by addition of fluorescent tags (such as green or yellow fluorescent protein) to enable visualisation within cells or organelles or to aid purification of the protein or cells expressing YcfD. Such modified polypeptides fall within the scope of the term “YcfD polypeptide”.

The YcfD polypeptide of the invention may be present in a partially purified or in a substantially isolated form. The polypeptide may be mixed with carriers or diluents, which will not interfere with its intended use and still be regarded as substantially isolated. The polypeptide may also be in a substantially purified form, in which case it will generally comprise at least about 90%, e.g. at least about 95%, 98% or 99%, of the proteins, polynucleotides, cells or dry mass of the preparation.

The YcfD polypeptide used in a method of the invention may be recombinant YcfD or naturally occurring YcfD. Naturally occurring YcfD may be obtained from any organism that produces a YcfD polypeptide. Preferably, recombinant YcfD is used especially where YcfD is required for purposes requiring large (>1 mg) amounts of protein such as for biophysical assays or for high throughput analyses. Recombinant YcfD may be produced using standard expression vectors that comprise nucleotide sequences encoding YcfD. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. (1989).

The YcfD polypeptide may be present in a cell. For example, methods of the invention may utilise cells that have been modified to express a YcfD polypeptide as defined herein. The YcfD may also be present in a cell extract or in a partially or substantially purified form.

A purified YcfD polypeptide may be obtained by introducing an expression vector comprising a polynucleotide encoding a YcfD polypeptide into a host cell.

Expression vectors are routinely constructed in the art and may for example involve the use of plasmid DNA and appropriate initiators, promoters, enhancers and other elements, such as for example polyadenylation signals which may be necessary and which are positioned in the correct orientation in order to allow full protein expression. Suitable vectors would be very readily apparent to those of skill in the art.

Promoter sequences may be inducible or constitutive promoters depending on the selected assay format. The promoter may be tissue specific. Thus the coding sequence in the vector is operably linked to such elements so that they provide for expression of the coding sequence (typically in a cell). The term “operably linked” refers to a juxtaposition wherein the components described are in a relationship permitting them to function in their intended manner.

The vector may be, for example, a plasmid, virus or baculovirus vector. The vector is typically adapted to be used in a bacterial cell, such as E. coli. The vector may have an origin of replication. The vector may comprise one or more selectable marker genes, for example an ampicillin resistance gene in the case of a bacterial plasmid or a resistance gene for a fungal vector. Vectors may be used to transfect or transform a host cell, for example, a bacterial host cell, fungal host cell, an insect host cell, a mammalian, e.g. human host cell or a baculovirus host cell. The bacterial host cell is preferably a strain of E. coli, for example BL21 (DE3).

Methods for introducing polypeptides and vectors into host cells are well known in the art, and include electroporation and heat shock techniques without limitation. Expression of the truncated polypeptide may then be achieved by culturing the host cells.

The YcfD polypeptide may be purified by lysing the host cells and extracting YcfD from the soluble fraction, for example by affinity purification, such as via an affinity tag fused to the truncated YcfD polypeptide. YcfD polypeptides may be purified by standard techniques known in the art. For example, where the polypeptide comprises a His tag, it may be purified using a his-binding resin by following the manufacturer's instructions (e.g. Novagen) or by other means such as ion exchange chromatography.

The methods of the present invention typically use a peptide containing an arginine-residue containing protein or peptide as a substrate (or binding agent) for the YcfD polypeptide. Short peptides can be used, for example peptides as short as 6 or 10 amino acids in length, typically at least 11 amino acids in length, such as 12, 13, 14, 15 or 16 amino acids in length up to much longer polypeptides and proteins, of at least 20, 30, 40, 50, 60, 70, 80, 90, 100, 110, 120, 130 or 136 amino acids in length. A full length protein which is a substrate for the YcfD polypeptide can be used, for example the L16 ribosomal protein of E. coli.

Any suitable peptide can be used, so long as the peptide contains an arginine residue (or analogue thereof) capable of hydroxylation by YcfD (or of binding to the active site of YcfD). The peptide may be modified, e.g. by the presence of group to facilitate assays such as a fluorescent group; Many such modifications are routinely used and described in the scientific literature. A number of peptides have been shown to be capable of hydroxylation by YcfD, such as those shown in Table 2. Peptides containing C-terminal arginine residues may be preferred for non-substrate specific hydroxylation of arginine.

In preferred aspects of the present invention, the peptide used in the assays is a substrate for YcfD in vivo, or a homologue, variant or fragment thereof. In particular, the present inventors have identified ribosomal protein L16 to be a substrate for YcfD.

Thus a preferred peptide containing arginine for use in accordance with the present invention is SEQ ID NO: 3 or a variant thereof or a fragment of either thereof. Typically, a variant thereof has an amino acid sequence having at least about 60% sequence identity, for example at least about 70% sequence identity, with SEQ ID NO: 3 over its entire length or over an active fragment thereof, typically greater than about 80% or 90%, such as about 95% or about 99% sequence identity.

Sequence identity may be calculated using any suitable algorithm. For example, the UWGCG Package provides the BESTFIT program can be used to infer homology (for example used on its default settings) (Devereux et al. (1984) Nucleic Acids Research 12, p 387-395). The PILEUP and BLAST algorithms can be used to infer homology or line up sequences (typically on their default settings), for example as described in Latched (1993) J. Mol. Evol. 36:290-300; Latched et al. (1990) J. Mol. Biol. 215:403-10.

Amino acid substitutions of SEQ ID NO: 3, or of a fragment thereof may be made, for example from about 1, 2 or 3 to about 10, 20 or 30 substitutions.

Conservative substitutions may be made, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

YcfD has been shown to hydroxylate arginine at position 81 of SEQ ID NO: 3. Thus a variant or homologue of SEQ ID NO: 3 includes an arginine equivalent to arginine at position 81 of SEQ ID NO: 3.

The assays of the present invention also include the use of fragments of SEQ ID NO: 3 or fragments of the variants thereof as defined above. Such fragments may be as short as 6 amino acids in length, typically at least 10, 11, 12 or 13 or 14 amino acids in length, and incorporate an arginine equivalent to arginine at position 81 of SEQ ID NO: 3. In peptides the arginine may be at the C-terminus.

The method of the invention may be used to identify a modulator of YcfD activity. The assay may be carried out in the presence of a test agent to determine whether the test agent is a modulator of YcfD activity. Such assays may use purified materials or be carried out in cells. Any suitable assay may be carried out to identify modulators of YcfD argininyl hydroxylase activity. A number of different examples of suitable assays are described below. Assays of the invention may be used to identify an agent which modulates, such as inhibits or activates, YcfD argininyl hydroxylase activity.

In a method of the invention YcfD activity may be assayed by monitoring oxygenase activity of aYcfD polypeptide in the presence of substrate. In some embodiments, the substrate is a ribosomal protein such as the E. coli ribosomal protein L16. In some embodiments, the YcfD polypeptide hydroxylates Arg-81 of the ribosomal protein L16, or fragment or analogue thereof. The substrate and YcfD polypeptide, and optionally the test agent, are typically contacted under conditions suitable for oxygenase (argininyl hydroxylase) activity.

Suitable co-substrates include oxygen, for example, dioxygen, and 2-oxoacids such as 2-oxogluterate (2-OG) or 2-OG analogues (such as 2-oxoadipate). Preferably, the co-substrate is 2-OG. In addition to oxygen or a 2-oxoacid, a reducing agent, such as ascorbate may also be used as a co-substrate. Thus, in a method according to the invention, the ribosomal protein or analogue or fragment thereof and YcfD polypeptide are contacted in the presence of Fe(II), oxygen and 2-oxoglutarate and optionally in the presence of a reducing agent.

Hydroxylation of the substrate may be assayed directly or indirectly. Such assays may employ techniques such as chromatography, NMR, MS or fluorescence spectroscopy. The co-substrate may be modified, e.g. 2-OG, consumed, e.g. oxygen or ascorbate, or produced, e.g. succinate or carbon dioxide, by YcfD.

In an assay to identify a modulator of YcfD activity, the components of the assay are contacted under conditions in which YcfD has argininyl hydroxylase activity both in the absence of the test agent and in the presence of the test agent so that the effect of the test agent on YcfD activity may be determined. The assay may also be used to detect agents that increase or decrease the activity of YcfD activity by assaying for increases or decreases in activity. Suitable assays have been described in the art for other 2-OG dependent oxygenases including the HIF hydroxyalses and histone demethylases.

Assays of the present invention may be used to identify inhibitors of oxygenase activity and are thus preferably, but not necessarily, carried out under conditions under which YcfD is active as an oxygenase (a argininyl hydroxylase) in the absence of the test agent. The YcfD oxygenase activity in the presence of the test agent is compared to YcfD oxygenase activity in the absence of the test substance to determine whether the test substance is an inhibitor of YcfD oxygenase activity. In the alternative, the assays may be used to look for promoters of YcfD oxygenase activity, for example, by looking for increased conversion of co-substrate and/or hydroxylation of substrates compared to assays carried out in the absence of a test substance. The assays may also be carried out under conditions in which oxygenase activity is reduced or absent, such as under hypoxic conditions, and the presence of or increased activity could be monitored under such conditions.

In medicinal applications, for example, it is often advantageous to modulate oxygenase activity of a single enzyme or group of enzymes. The assays of the invention may also be used to identify inhibitors or activators that are specific for prokaryotic hydroxylases, such as YcfD (or homologues of YcfD) and which do not have activity or are less active with other 2-OG oxygenases, including eukaryotic enzymes, such as human 2OG oxygenases. Conversely, the assays of the invention may be used to identify inhibitors or activators specific for one or more 2-OG dependent oxygenase which do not inhibit YcfD activity. Human 2-OG oxygenases that may be tested in such a method of the invention are listed in Table 1. Such 2OG oxygenases include, but are not limited to: argininyl, prolyl, asparaginyl and arginyl demethylases, hypoxia inducible factor (HIF) asparaginyl or prolyl hydroxylases, including FIH, PHD1, PHD2 and PHD3, AlkB, ABH1, ABH2, ABH3, procollagen prolyl and argininyl hydroxylases, methyl arginine demethylases, Mina53, the fat mass and obesity protein, the epidermal growth factor hydroxylases, AlkB, TauD, and other 2-OG oxygenases that have been characterized as Jmj domain proteins according to the SMART database including, but not limited to argininyl demethylases.

TABLE 1
List of known or predicted human 2OG oxygenases

| Sub-family | Gene Id | Protein description |
|---|---|---|
| ASPH | 444 | Aspartyl/asparaginyl beta-hydroxylase (Aspartate beta-hydroxylase) (ASP beta-hydroxylase) |
| | | (Peptide-aspartate beta-dioxygenase) |
| ASPHD2 | 57168 | hypothetical protein LOC57168 |
| ASPHD1 | 253982 | hypothetical protein LOC253982 |
| C17orf101 | 79701 | PKHD domain-containing transmembrane protein C17orf101 |
| LEPRE1 | 64175 | leucine proline-enriched proteoglycan (leprecan) 1 |
| LEPRE1-like | 55214 | leprecan-like 1 |
| LEPRE2 | 10536 | leprecan-like 2 |
| P4H TM | 54681 | hypoxia-inducible factor prolyl 4-hydroxylase isoform a, transmembrane (endoplasmic reticulum) |
| P4HA3 | 283208 | procollagen-proline, 2-oxoglutarate 4-dioxygenase (proline 4-hydroxylase), alpha polypeptide III |
| P4HA1 | 5033 | procollagen-proline, 2-oxoglutarate 4-dioxygenase (proline 4-hydroxylase), alpha polypeptide I |
| P4HA2 | 8974 | procollagen-proline, 2-oxoglutarate 4-dioxygenase (proline 4-hydroxylase), alpha polypeptide II |
| PLOD3 | 8985 | procollagen-lysine, 2-oxoglutarate 5-dioxygenase 3 |
| PLOD1 | 5351 | procollagen-lysine, 2-oxoglutarate 5-dioxygenase 1 precursor (Lysyl hydroxylase 1) (LH1) |
| PLOD2 | 5352 | procollagen-lysine, 2-oxoglutarate 5-dioxygenase 2 isoform a |
| JMJD4 | 65094 | JMJD4 isoform 1 |
| JMJD6 | 23210 | Phosphatidylserine receptor JMJD6 isoform 1 |
| JMJD5 | 79831 | Hypothetical protein FLJ13798 |
| JMJD8 | 339123 | Hypothetical LOC339123 |
| TYW5/C2orf60 | 129450 | C2orf60 chromosome 2 open reading frame 60 |
| FIH | 55662 | Hypoxia-inducible factor 1 alpha inhibitor (Hypoxia-inducible factor asparagine hydroxylase) |
| | | (Factor inhibiting HIF-1) (FIH-1) |
| PASS1/HSPBAP1 | 79663 | PASS1 |
| JMJD7/PLA2gIVB | 8681 | phospholipase A2, group IVB |
| NO66 | 79697 | chromosome 14 open reading frame 169 |
| MINA53B | 84864 | MYC induced nuclear antigen, isoform 2 |
| JMJD3/KDB6B | 23135 | jumonji domain containing 3 |
| UTX/KDM6A | 7403 | ubiquitously transcribed tetratricopeptide repeat, X chromosome |
| UTY | 7404 | tetratricopeptide repeat protein isoform 1 |
| JARID1B/PLU-1/KDM5B | 10765 | Jumonji, AT rich interactive domain 1B (RBP2-like) |
| JARID1A/RBBP2/KDM5A | 5927 | retinoblastoma binding protein 2 |
| JARID1C/SMCX/KDM5C | 8242 | Smcx homolog, X chromosome |
| JARID1D/SMCY/KDM5D | 8284 | Smcy homolog, Y-linked |
| JMJD2A/JHDM3A/KDM4A | 9682 | jumonji domain containing 2A |
| JMJD2C/GASC1/KDM4C | 23081 | jumonji domain containing 2C |
| JMJD2B/KDM4C | 23030 | jumonji domain containing 2B |
| JMJD2D/KDM4D | 55693 | jumonji domain containing 2D |
| JMJD2E/KDM4E | | jumonji domain containing 2E; pseudogene |
| FBXL10/JHDM1B/KDM2B | 84678 | F-box and leucine-rich repeat protein 10 |
| FBXL11/JHDM1A/KDM2A | 22992 | F-box and leucine-rich repeat protein 11 |
| KIAA1718/JHDM1D | 80853 | KIAA1718 protein |
| PHF8/KIAA1111 | 23133 | PHD finger protein 8 |
| PHF2/JHDM1E/GRC5 | 5253 | PHD finger protein 2 isoform a |
| HR | 55806 | Hairless |
| JMJD1A/KDM3A/TSGA | 55818 | jumonji domain containing 1A |
| JMJD1B/KDM3B/5qNCA | 51780 | jumonji domain containing 1B |
| JMJD1CA/TRIP8/KIAA1380 | 221037 | jumonji domain containing 1C isoform a |
| JARID2/JMJ | 3720 | JARID2 original Jumonji protein - missing iron binding residue |
| PHD1 | 112398 | HIF prolyl-4-hydroxylase, N-terminal domain disordered |
| PHD2 | 54583 | HIF prolyl-4-hydroxylase, N-terminal MYND |
| PHD3 | 112399 | HIF prolyl-4-hydroxylase, No N-terminal domain |
| ABH1 | 8846 | Alkylated DNA repair protein alkB homolog ABH1 |
| ABH2 | 121642 | similar to hypothetical protein 9530023G02 ABH2 |
| ABH3 | 221120 | hypothetical protein LOC221120 ABH3 |
| ABH4 | 54784 | hypothetical protein LOC54784 ABH4 |
| ABH5 | 54890 | hypothetical protein LOC54890 ABH5 |
| ABH6 | 84964 | probable alpha-ketoglutarate-dependent dioxygenase ABH6 isoform 1 |
| ABH7 | 84266 | probable alpha-ketoglutarate-dependent dioxygenase ABH7 precursor |
| ABH8 | 91801 | 5-methoxycarbonylmethyluridine hydroxylase - woble position of tRNA, C-terminal |
| | | Ado-Met-MTase domain |
| FTO | 79068 | Fat mass and Obesity associated DNA demethylase |
| TET1 | 80312 | methylcytosine dioxygenase TET1, CXXC finger 6 |
| TET2 a | 54790 | methylcytosine dioxygenase TET2 isoform a |
| TET2 b | 54790 | methylcytosine dioxygenase TET2 isoform b |
| TET3 | 200424 | methylcytosine dioxygenase TET3 |
| PAHX | 5264 | phytanoyl-CoA hydroxylase precursor |
| PHYHD1 | 254295 | PHYHD1 protein |
| GBBH | 8424 | gamma-butyrobetaine hydroxylase |
| TMLH | 55217 | trimethyllysine hydroxylase, epsilon |

The present invention also provides a method for identifying a selective inhibitor of YcfD (or YcfD homologue), or an inhibitor that is selective for another 2OG-oxygenase over YcfD. This method comprises: (i) contacting a protein involved in RNA biochemistry/metabolism, processing or splicing, or fragment thereof comprising a arginine residue, with a YcfD polypeptide in the presence of a test agent and determining whether the protein or fragment thereof is hydroxylated; (ii) determining whether the test agent modulates activity of a 2-OG dependent oxygenase other than YcfD, thereby determining whether the test agent selectively modulates YcfD activity or selectively modulates activity of the 2-OG dependent oxygenase other than YcfD.

Oxygenase activity of the 2-oxoglutarate dependent oxygenase other than YcfD may be determined by contacting a substrate of the 2-OG dependent oxygenase with the 2-OG dependent oxygenase in the presence of a test agent and determining whether the substrate is hydroxylated or demethylated or otherwise oxidised. In an assay to identify a selective inhibitor of YcfD, or another oxygenase, different substrates may be used for YcfD and for the other oxygenase(s).

Alternatively, oxygenase activity of the 2-OG dependent oxygenase other than YcfD may be determined in the absence of a prime substrate (i.e. a non 2-OG substrate). This enables selective inhibitors to be identified when the prime substrate of one or more of the enzymes being tested is unknown. In this embodiment, generally it will be one or more of the enzymes that it is wished not to inhibit that is an enzyme that has an unknown substrate. The effect of a test agent on activity of an oxygenase may be determined in the absence of a substrate by determining whether or not the test agent affects, for example inhibits or stimulates, the rate of turnover of 2-OG by the oxygenase.

Thus, the invention also provides methods for screening for compounds that do not inhibit YcfD. Such compounds are of use with respect to developing inhibitors that are selective for 2-OG oxygenases other than YcfD.

The assays of the invention may also be used to identify inhibitors or activators, which are specific for YcfD activity at a particular substrate or residue within a substrate.

Such selectivity screens may be used to identify selective inhibitors of YcfD or selective inhibitors of other enzymes, i.e. inhibitors that are more potent inhibitors of YcfD activity than of activity of the other enzyme or inhibitors that are less potent inhibitors of YcfD activity than of activity of the other enzyme. Where the inhibitor is a selective inhibitor of YcfD activity it may have no effect on the activity of the other enzyme or may exhibit only a low level of inhibition, such as less than about 50% inhibition on activity of the other enzyme. Where the inhibitor is a selective inhibitor of the activity of the enzyme other than YcfD, it may have no effect on the activity of YcfD or may exhibit only a low level of inhibition, such as less than about 50% inhibition of YcfD activity.

The selectivity screens may be carried out with purified enzymes, partially purified enzymes (such as in crude cell lysates) or in cells.

The invention provides for the use of selective inhibitors in the manufacture of a medicament for the treatment of a condition associated with altered, i.e. enhanced or reduced YcfD oxygenase activity.

The precise format of any of the assay or screening methods of the present invention may be varied by those of skill in the art using routine skill and knowledge. The skilled person is well aware of the need to additionally employ appropriate controlled experiments. The assays of the present invention may involve monitoring for hydroxylation of the substrate, monitoring for the utilisation of substrates and co-substrates, monitoring for the production of the expected products between the enzyme and its substrate. Assay methods of the present invention may also involve screening for the direct interaction between components in the system. Alternatively, assays may be carried out which monitor for downstream effects mediated by the substrate, such as substrate mediated transcription using suitable reporter constructs or by monitoring for the upregulation of genes or alterations in the expression patterns of genes known to be regulated directly or indirectly by the substrate.

Various methods for determining oxygenase activity either directly or indirectly are known in the art. Any suitable method may be used for determining 2-OG dependent oxygenase activity of YcfD such as by substrate or co-substrate utilisation, product appearance such as peptide hydroxylation (or demethylation for some 2-OG oxygenases) or down-stream effects mediated by hydroxylated (or demethylated or non-hydroxylated products for some 2-OG oxygenases).

The substrate, enzyme and potential inhibitor compound may be incubated together under conditions which, in the absence of inhibitor provide for hydroxylation (or demethylation for some 2-OG oxygenases) of the substrate, and the effect of the inhibitor may be determined by determining hydroxylation (or demethylation for some 2-OG oxygenases) of the substrate. This may be accomplished by any suitable means. Small polypeptide or polynucleotide substrates may be recovered and subjected to physical analysis, such as mass spectrometry, radiography or chromatography, or to functional analysis. Such methods are known as such in the art and may be practiced using routine skill and knowledge. For example, the LC-MS assay described in the Examples may be used. Determination may be quantitative or qualitative. In both cases, but particularly in the latter, qualitative determination may be carried out in comparison to a suitable control, e.g. a substrate incubated without the potential inhibitor.

In alternative embodiments, reporter constructs may be provided in which promoters mediated by a substrate are provided operably linked to a reporter gene. Any suitable reporter gene could be used, such as for example enzymes which may then be used in colorometric, fluorometric, fluorescence resonance or spectrometric assays.

In the assay methods described herein, typically the YcfD polypeptide and the substrate are contacted in the presence of a co-substrate, such as oxygen and/or a 2-oxoacid, such as 2-OG, and/or dioxygen. Hydroxylase activity may be determined by determining turnover of one or more of the co-substrates, such as oxygen, 2-OG and/or ascorbate. This may be achieved by determining the presence and/or amount of reaction products, such as hydroxylated substrate, carbon dioxide or succinic acid. The amount of product may be determined relative to the amount of substrate. For example, in such embodiments the product measured may be hydroxylated peptide or protein. In the case of protein the extent of hydroxylation may also be determined in cells, e.g. by the use of appropriate antibodies or by mass spectrometry. For example, the extent of hydroxylation may be determined by measuring the amount of hydroxylated peptide/protein, succinate, carbon dioxide, or formaldehyde generated in the reaction, or by measuring the depletion of 2-OG or dioxygen. Methods for monitoring each of these are known in the scientific literature, for example in Myllyharju et al. (1991) EMBO J. 16(6): 1173-1180 or as in Cunliffe et al. (1986) Biochem. J. 240: 617-619. An assay that measures oxygen consumption such as that described by Ehrismann et al. Biochem J. (2007) may be used. In addition, an enzyme activity assay that measures ¹⁴CO₂ generated from the decarboxylation of [¹⁴C] 2-OG coupled to hydroxylation (Kivirikko K I, Myllyla R. Methods Enzymol (1982) may also be used. (Dissolved oxygen electrodes, exemplified by but not limited to a “Clarke-type” electrode or an electrode that uses fluorescence quenching, may be used to follow the consumption of oxygen in an assay mixture.) Use of ion-exchange chromatography to separate [¹⁴C]-succinic acid and [5-¹⁴C]-2-OG or separation using 2,4-dinitrophenylhydrazine to precipitate [5-¹⁴C]-2-OG may also be used. Measuring conversion of [5-¹⁴C]-2-OG to [¹⁴C]-succinic acid Kanelakis K C, Palomino H L, Li L, et al. J Biomol Screen (2009) may also be used. The formation of hydroxylated peptide fragment can be determined directly, e.g. by using either LC/MS analysis, Li D, Hirsila M, Koivunen P, et al. J Biol Chem (2004), or matrix-assisted laser desorption ionization, time-of-flight mass spectrometer or by other assay monitoring hydroxylation. Monitoring the consumption of a reducing agent such as potassium ferrocyanide (replacing ascorbate) FibroGen, Inc. WO2005118836; 2007 may be used. Antibody based methods may also be used by employing an antibody selective for a hydroxylaetd product. Antibody based methods may be enhanced such that they are more efficient for modulator screening, e.g. by use of homogenous time resolved fluorescence (HTRF) methods which measure the energy transfer between a labelled dye (e.g., via biotin—streptavidin complex) to hydroxyl-arginine peptide fragment substrate, and europium, which is tagged to a hydroxyl-arginine specific antibody similar to methods described in Dao J H, Kurzeja R J M, Morachis J M, et al. Anal Biochem (2009).

The amount of unused 2-OG may be determined by derivatisation, by chemical reagents, exemplified by but not limited to hydrazine derivatives and ortho-phenylene diamine derivatives, to give indicative chromophores or fluorophores that can be quantified and used to indicate the extent of hydroxylation of the substrate. Suitable methods are described in McNeill et al. (2005) (Anal. Biochem. 366:125-131). The fluorescent product of the reaction of ortho-phenylenediamine (OPD) with the α-ketoacid motif of 2-OG is 3-(2-Carboxyethyl)-2(1H)-quinoxalinone. This fluorescent product can be readily detected by standard equipment such as that manufactured by for example Molecular Devices, Tecan, BMG Labtechnologies, Jasco and Perkin Elmer and there is extensive precedent demonstrating that the production of fluorescent products can be used in high-throughput screens.

The fluorescent product is generally detected with the excitation filter set as from about 300 nm to about 400 nm, preferably from about 335 to about 345 nm, most preferably at about 340 nm. The emission filter is generally at from about 400 to about 450 nm, preferably from about 415 to about 425 nm, most preferably at about 420 nm. The nature of the fluorescent product can be tuned by modifying the nature of the derivatisation reagent used. For example, the sensitivity of the method may be increased by using either 1,2-dimethoxy-4,5-diaminobenzene, or 1,2-methylenedioxy-4,5-diaminobenzene.

The precise format of any of the screening or assay methods of the present invention may be varied by those of skill in the art using routine skill and knowledge. The skilled person is well aware of the need to additionally employ appropriate control experiments.

Other components may be added to the assay mixtures. For example, a reducing agent such as ascorbate, a thiol such as dithiothrietol (DDT), (3-mercaptoethanol, tris(2-carboxyethyl)phosphine hydrochloride (TCEP), N-acetylcysteine or phenol may be added to the assay to help maintain enzyme structure and/or catalase may be added to destroy any H₂O₂ that might be produced. However, the assay will work in the absence of a reducing agent or catalase.

Assays are typically carried out at a temperature of from about 25° C. to about 40° C., for example at a temperature of from about 30° C. to about 39° C., or from about 35° C. to about 38° C. or about 37° C. The pH of the assay mixture is typically between about pH 7 to about pH 9, for example from about pH 7.5 to about pH 8. Suitable buffers, such as Tris or HEPES, may be used to maintain the pH of the assay mixture.

Typically, assays are carried out under normoxic conditions, but may be carried out at oxygen concentrations above atmospheric levels. The assay may also be carried out under conditions in which hydroxylation or oxidation is reduced or absent, such as under hypoxic conditions, in order to detect modulation of oxygenase activity by an agent which enhances hydroxylation/oxidation.

Alternatively, the end-point determination may be based on conversion of the substrate or substrate fragments (including synthetic and recombinant peptides or nucleic acids) derived from the polypeptide or nucleic acid substrate into detectable products. Substrates may be modified to facilitate the assays so that they can be rapidly carried out and may be suitable for high throughput screening.

For example, reverse phase HPLC(C-4 octadecylsilane column), as exemplified herein, may be used to separate starting synthetic peptide substrates for subtraters from the products. Modifications of this assay or alternative assays for oxygenase activity may employ, for example, mass spectrometric, spectroscopic, and/or fluorescence techniques as are well known in the art (Masimirembwa C. et al. Combinatorial Chemistry & High Throughput Screening (2001) 4 (3) 245-263, Owicki J. (2000) J. Biomol. Screen. 5 (5) 297-305, Gershkovich A et al. (1996) J. Biochem. & Biophys. Meths. 33 (3) 135-162, Kraaft G. et al. (1994) Meths. Enzymol. 241 70-86). Fluorescent techniques may employ versions of the substrate modified in such as way as to carry out or optimise spectroscopic or fluorescence assays.

Binding of a molecule, such as an antibody, which discriminates between the hydroxylated and non-hydroxylated forms of a peptide or protein may be assessed using any technique available to those skilled in the art, which may involve determination of the presence of a suitable label.

Assay methods of the present invention may also take the form of an in vivo assay or an assay carried out on ex vivo cells from an animal, such as a mammal (including human) or an insect. The assay may be performed in a cell line such as a yeast or bacterial strain or an insect or mammalian cell line in which the relevant polypeptides or peptides are expressed from one or more vectors introduced into the cell. Alternatively, the assay may be carried out on a prokaryotic cell that expresses endogenous YcfD or in which YcfD is over expressed.

The present invention further provides a method for introducing hydroxyarginine residues into peptides or proteins. In particular a protein or peptide containing an arginine residue may be contacted with a YcfD polypeptide as described herein, in order to hydroxylate the arginine residue. Hydroxylation of arginine residues may be used for example to increase the stability of the peptide or protein. Hydroxylation of arginine may also be used to modify the activity of the protein. Hydroxylation of arginine may also be used to introduce a glycosylation site into the peptide or protein.

The invention further provides a method for identifying a modulator of protein translation, the method comprising contacting a cell which expresses YcfD with a test agent and determining whether the test agent modulates YcfD-mediated regulation of protein translation, either by modulating YcfD (or YcfD homologue) activity or by modulating translation (including translational accuracy) in a hydroxylation dependent manner relating to the YcfD (or YcfD homologue) catalysed hydroxylation of a ribosomal protein.

In one embodiment YcfD may be over-expressed in the cell. YcfD may be over-expressed in a cell in vitro or in vivo by any suitable method, typically by introducing an expression vector encoding a YcfD polypeptide into the cell. Protein translation (or translation accuracy) may be monitored in the cell over-expressing YcfD and compared to protein translation in a control cell that does not over-express YcfD. The cell over-expressing YcfD may be contacted with a test agent and protein translation may be monitored in the presence of the test agent. By comparing translation observed in the presence and absence of the test agent and in the presence and absence of YcfD over-expression, it may determined whether the test agent modulates YcfD-mediated regulation of protein translation. Levels of YcfD catalysed hydroxylation in cells may be determined by use of antibodies or by mass spectrometric methods as routinely used in proteomic analyses.

In another embodiment, YcfD may be under-expressed in the cell. YcfD may be under-expressed in a cell in vitro or in vivo by any suitable method, for example by using RNAi technology to knock down the YcfD protein. Protein translation may be monitored in the cell under-expressing YcfD and compared to protein translation in a control cell that does not under-express YcfD. The cell under-expressing YcfD may be contacted with a test agent and protein translation may be monitored in the presence of the test agent. By comparing the protein translation observed in the presence and absence of the test agent and in the presence and absence of YcfD under-expression, it may determined whether the test agent modulates YcfD-mediated regulation of protein translation.

Methods for monitoring protein translation or translation accuracy are well known in the art. For example, protein translation may be monitored using a reporter construct. Thus, in a method for identifying a modulator of protein translation according to the invention, the cell may comprise a protein translation reporter construct and the method may comprise determining whether YcfD-mediated regulation of protein translation of the reporter construct is modulated by the test agent.

Agents, which may be screened using the assay methods described herein, may be natural or synthetic chemical compounds used in drug screening programmes. Extracts of plants, microbes or other organisms, which contain several, characterised or uncharacterised components may also be used.

Combinatorial library technology (including solid phase synthesis and parallel synthesis methodologies) can provide an efficient way of testing a potentially vast number of different substances for ability to modulate an interaction. Such libraries and their use are known in the art, for all manner of natural products, small molecules and peptides, among others. The use of peptide libraries may be preferred in certain circumstances. Various commercial libraries of compounds are also available. There are computational methods for screening these libraries (processes sometimes referred to as virtual screening) that can identify lead structures for inhibition.

Potential inhibitor compounds (i.e. antagonists) may be polypeptides, peptides, small molecules such as molecules from commercially available libraries, including combinatorial libraries, or the like. The peptide may be a cyclic peptide. Small molecule compounds, which may be used, include 2-OG analogues, or substrate analogues, which inhibit the action of the enzyme. Small molecule compounds, and other types of compound, that may be used include all known 2-OG oxygenase inhibitors such as those already known to inhibit HIF hydroxylases (see for example WO03/080566, WO02/074981, WO2007/146483, WO2007136990, WO2007/103905, WO2007/150011, US2007/0299086, US2007/0249605 and US2007/0213335), procollagen prolyl hydroxylases, and histone demethylases (for which the output of high throughput screening data is publicly available—see e.g. King et al. PLoS ONE 5(11): e15535. doi:10.1371/journal.pone.0015535 and associated material).

Potential promoting agents may be screened from a wide variety of sources, particularly from libraries of small compounds, which may be commercially available. Candidate compounds to be screened, may include 2-OG analogues, compounds that chelate iron or known families of 2OG oxygenases inhibitors.

Since naturally occurring compounds, including TCA cycle intermediates such as fumarate and succinate, are known inhibitors of 2-OG oxygenases they may inhibit YcfD (or YcfD homologues), possibly in a manner that is of physiological relevance, including in some cancers where fumarate is known to be upregulated as a consequence of the Warburg effect.

A test compound which increases, potentiates, stimulates, disrupts, reduces, interferes with or wholly or partially abolishes hydroxylation of the substrate and which may thereby modulate activity, may be identified and/or obtained using the assay methods described herein.

Agents which increase or potentiate hydroxylation (i.e. agonists), may be identified and/or obtained under conditions which, in the absence of a positively-testing agent, limit or prevent hydroxylation. Such agents may be used to potentiate, increase, enhance or stimulate the oxygenase activity of YcfD.

In various aspects, the present invention provides an agent or compound identified by a screening method of the invention to be a modulator of YcfD oxygenase activity e.g. a substance which inhibits or reduces, increases or potentiates the activity of YcfD.

The test agent may compete with 2-OG or a YcfD substrate at the YcfD active site and/or binds to the active site of YcfD or to metal at the YcfD active site. The test agent may comprise a metal ion such as, but not limited to, manganese, cobalt, zinc or nickel ions as inhibitors or iron (II), iron (III) as activators. Alternatively, the mode of inhibition may be via competition with the substrate or by an allosteric interaction.

The test agent may be a reducing agent. Reducing agents typically act as activators of 2-OG oxygenase activity, typically in vitro. An activator of oxygenase activity may be any species that increases oxygenase activity of a YcfD polypeptide either in vitro or in vivo. Reducing agents that may be used include ascorbate and analogues of ascorbate and reducing agents of the thiol chemical families, such as dithiothreitol or phosphine (e.g. triscarboxyethylphosphine).

Following identification of a modulator, the substance may be purified and/or investigated further (e.g. modified) and/or manufactured. A modulator may be used to obtain peptidyl or non-peptidyl mimetics, e.g. by methods well known to those skilled in the art and discussed herein. A modulator may be modified, for example to increase selectively, as described herein. It may be used in a therapeutic context as discussed below.

For therapeutic treatment, the modulator may be alone or used in combination with any other therapeutically active substance or treatment including but not limited to metal ions or succinate or fumarate (Chen et al. J Biol Chem 2010).

The compounds which are acids can be present in the form of salts, such as sodium salts. The compounds may also be present in the form of derivatives such as the dimethyl ester, diethyl ester, monoethyl ester or di- or mono-amide, or other prodrug form rendering suitable pharmokinetic properties. In certain instances these derivatives may be preferred, for example when inhibition of the enzyme within a cell of an organism is required.

Compounds which modulate 2-OG oxygenases may be useful as agents of the invention, for example, in the treatment of disorders as described herein, or may be used as test substances in an assay of the invention. The test compound may be known to act as an inhibitor of a 2-OG oxygenase other than YcfD. For example, the test agent may be a described inhibitor of procollagen prolyl hydroxylase, hypoxia inducible factor, prolyl and asparaginyl hydroxylases, collagen prolyl hydroxylase, gibberellin C-20 oxidase, a nucleic acid demethylase such as AlkB or a human AlkB homologue, a protein demethylase, such as a tri-, di-, mono-methyl lysine or arginine residue demethylase, another human or animal 2OG oxygenase involved in metabolism or regulation, or a plant 2-OG hydroxylase. Many inhibitors of 2OG oxygenases are known in particular for human prolyl hydroxylases and hsitoen demethylases. N-Oxaloglycine and its derivatives are one such examples, but there are many others, which one of skillin the art of oxygenases may test as YcfD inhibitors. Glycine or alanine derivatives and 2-oxoacid analogues may also be used.

Compounds which modulate 2-OG oxygenases, and families of such compounds, are known in the art, for example in Aoyagi et al. (2002) Hepatology Research 23 (1): 1-6, Aoyagi et al. (2003) Free Radical Biology and Medicine 35:410 Suppl. 1, Philipp et al. (2002) Circulation 106 (19): 1344 Suppl. S, Ivan et al. (2002) PNAS USA 99 (21): 13459-13464, Nwogu et al. (2001) Circulation 104 (18): 2216-2221, Myllyharju and Kivirikko (2001) Ann Med 33 (1): 7-21, Ohta et al. (1984) Chemical and Pharm Bulletin 32 (11): 4350-4359, Franklin et al. (2001) Biochem J. 353: 333-338, Franklin (1997) Int J. Biochem Cell Biol 29 (1): 79-89, Dowell et al. (1993) Eur J Med Chem 28 (6): 513-516, Baader et al. (1994) Biochem J. 300: 525-530, Baader et al. (1994) Eur J Clin Chem and Clin Biol 32 (7): 515-520, Bickel et al. (1998) Hepatology 28 (2): 404-411, Bickel et al. (1991) J. Hepatology 13: S26-S34 Suppl. 3, U.S. Pat. No. 6,200,974, U.S. Pat. No. 5,916,898, US Patent Applications 2003-0176317, 2003-0153503 and 2004-0053977, WO 02/074981, WO 03/080566, WO 04/035812, Cunliffe et al. (1992) J. Med. Chem. 35:2652-2658, Higashide et al. (1995) J. Antibiotics 38:285-295, Cunliffe et al. (1986) Biochem. J. 239(2):311-315, Franklin et al. (1989) Biochem. J. 261(1):127-130, Friedman et al. (2000) PNAS USA 97(9):4736-4741, Wu et al. (1999) J. Am. Chem. Soc. 121(3): 587-588, DE-A-3818850, Wang et al. (2001) Biochemistry US:15676-15683 and Lerner et al. (2001) Angew Chem. Int. Edit. 40:4040-4041. Rose et al. J Med Chem (2008), Rose et al. J Med Chem (2010), Conjeo-Garcia et al. Bioorg Med Chem. Lett. (2010), Banjeri et al. Chem Commun (2005), Hewitson et al. J Biol Chem (2007), McDonough et al. J Am Chem Soc (2005), Mecinovic et al. Bioorg Med Chem Lett (2009), Lienard et al. Chem Commun (2008), Hamada et al. J Med Chem (2010), Simkhovich et at Biochem Pharmacol (1988).

Suitable compounds are disclosed in WO03/080566, WO02/074981, WO2007/146483, WO2007136990, WO2007/103905, WO2007/150011, US2007/0299086, US2007/0249605, WO2009/074498 and US2007/0213335. Other suitable compounds include inhibitors of HIF hydroxylase. HIF hydroxylase inhibitors are disclosed in United States Patent Application Publication Nos: 20070042937, 20060276477, 20060270699, 20060258702, 20060258660, 20060251638, 20060183695, 20060178317 and 20060178316 and in International Patent Application Publication Nos: WO2007/070359, WO2008/002576, WO2007/103905, WO2005118836, WO2003049686, WO2003053997, US20060276477, US20070292433, US20070293575, WO2004108121. US20060251638, WO2004052285, WO2005011696, WO2005034929, WO2004052284, WO2006099610, WO2007097929, WO2009075824, WO2009075826, WO2006138511, WO2009058403, WO2009075826, WO2006138511, WO2009058403, WO9921860, WO2006094292, WO2007090068, WO2007115315, WO2009073669, WO2009089547, WO2009100250, WO2010056767, WO2010022240, WO2004052313, WO2007038571, WO2007103905, WO2007136990, WO2009039323, WO2009039321, WO2009039322, WO2010022307, WO2009070644, WO2009073497, WO2009134850, WO2009134847, WO2007150011, US20080171756, WO2008089052, WO2009158315, WO2010025087, WO2009049112, WO2009086044, WO2010022308, WO2010059549, WO2010059552, WO2010059555, WO2007070359, WO2008076425, WO2008137084, WO2008076427, WO2008130508, WO2008130600, WO2008137060, WO2006114213, WO2008067874, DE102007044032, WO2008049538, DE102007048447, DE102007049157, WO2008067871, US20090269420, WO2008130527, WO2009108496, WO2009108497, WO2009108499, WO2008144266, WO2009137291, WO2009117269, WO2009134750, WO2009134754, US20080124740, US20070299086, WO2009037570, WO2010018458, WO2009016812.

Other suitable compounds include compounds of formula (I):

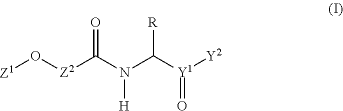

wherein
Y² is selected from —OR′ and —NR′R″ wherein R′ is hydrogen, or unsubstituted C₁₋₄ alkyl and R″ is hydrogen, hydroxy or unsubstituted C₁₋₄ alkyl;
Y¹ is selected from —C—, —S— and —S(O)—;
Z² is selected from —C(O)— and —NR″— wherein R″ is selected from hydrogen, hydroxy or unsubstituted C₁₋₄ alkyl;
Z¹ is selected from hydrogen and unsubstituted C₁₋₄ alkyl; and
R is a side chain of a naturally occurring amino acid.

Preferably Y¹ is —C— and Y² is —OH or —NH₂. Most preferably Y¹ is —C— and Y² is —OH.

Preferably Z² is —C(O)— or —NR″— wherein R″ is hydrogen, methyl or ethyl. More preferably Z² is —C(O)— or —NH—. Preferably Z¹ is hydrogen, methyl or ethyl, more preferably hydrogen. Most preferably Z² is —C(O)— and Z¹ is hydrogen, methyl or ethyl.

Preferably R is a side chain of alanine, valine, leucine or phenylalanine. Preferably R is a side chain of valine, leucine or phenylalanine More preferably R is a side chain of phenylalanine, i.e. —CH₂Ph.

L-stereoisomers or D-stereoisomers of these compounds may be used.

An exemplary synthetic scheme used to obtain test compounds of formula (I) is shown below in Scheme 1. Here an amino acid is reacted with an oxalyl chloride in order to produce a compound of formula (I). In this scheme the amino acid used is phenylalanine, although it will be apparent that the same general reaction will occur with other amino acids. The first reaction yields a protected compound of the invention (the dimethyl ester form). The diacid form is easily generated through reaction with aqueous sodium hydroxide.

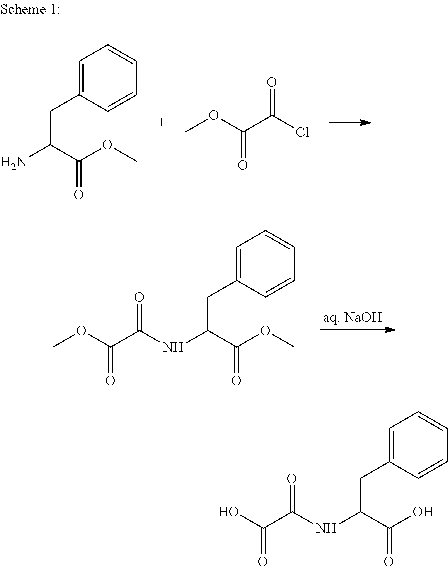

Compounds in which X is —O— or —S— or Z is other than —CO—CO—OH may by synthesised as described in Mole et al. (2003) Bioorg. Med. Chem. Lett. 13, 2677-2680 and Cunliffe et al. J. Med. Chem. (1992) 35 2652-2658.

Krebs cycle intermediates such as succinate and fumarate act as inhibitors of FTO demethylase activity. Therefore analogues of succinate and fumarate may be used to inhibit FTO activity.

In particular, the inventors have shown that the following compounds are inhibitory of YcfD argininyl hydroxylase activity: an N-oxalyl amino acid such as N-oxalylglycine (NOG) or a derivative thereof, a glycine or alanine derivative, a 2-oxoacid analogue, a flavonoid or flavonoid derivative such as genistein, pyridine-2,4-dicarboxylic acid, fumarate, succinate, FG0041, FG2216 or LBE-6.

Enhancers of YcfD activity include chelating agents such as pyridine-2,5-dicarboxylic acid and pyridine-2,6-dicarboxylic acid.

The present invention provides the use of an inhibitor or activator of 2-OG oxygenase activity to modulate argininyl hydroxylation of ribosomal protein by YcfD.

A compound, substance or agent which is found to have the ability to affect the oxygenase (argininyl hydroxylase) activity of YcfD has therapeutic and other potential in a number of contexts, as discussed.

The modulator of YcfD argininyl hydroxylase activity, may be a known inhibitor of a 2OG-dependent oxygenase, such as an N-oxalyl amino acid such as N-oxalylglycine (NOG) or a derivative thereof, a glycine or alanine derivative, a 2-oxoacid analogue, a flavonoid or flavonoid derivative such as genistein, pyridine-2,4-dicarboxylic acid, fumarate, succinate, FG0041, FG2216 or LBE-6. The inhibitor may be a selective inhibitor of YcfD activity compared to other 2-OG oxygenases.

An agent identified using one or more primary screens (e.g. in a cell-free system) as having ability to modulate oxygenase activity may be assessed further using one or more secondary screens.

Generally, an agent, compound or substance which is a modulator according to the present invention is provided in an isolated and/or purified form, i.e. substantially pure. This may include being in a composition where it represents at least about 90% active ingredient, more preferably at least about 95%, more preferably at least about 98%. Any such composition may, however, include inert carrier materials or other pharmaceutically and physiologically acceptable excipients, such as those required for correct delivery, release and/or stabilisation of the active agent.

The invention further provides compounds obtained by assay methods of the present invention, and compositions comprising said compounds, such as pharmaceutical compositions wherein the compound is in a mixture with a pharmaceutically acceptable carrier or diluent. Examples of suitable carriers or diluents are given in, for example, “Harrison's Principles of Internal Medicine”. The carrier may be liquid, e.g. saline, ethanol, glycerol and mixtures thereof, or solid, e.g. in the form of a tablet, or in a semi-solid form such as a gel formulated as a depot formulation or in a transdermally administrable vehicle, such as a transdermal patch.

The invention further provides a method of treatment which includes administering to a patient an agent which modulates YcfD oxygenase activity. Such agents may include inhibitors of YcfD oxygenase activity.

A therapeutically effective amount of an agent is typically administered to a subject in need thereof. Typically such agents can be used as ant-microbial agents, for example as antibiotics.

In various further aspects, the present invention thus provides a pharmaceutical composition, medicament, drug or other composition for such a purpose, the composition comprising one or more agents, compounds or substances as described herein, including inhibitors of YcfD oxygenase activity, the use of such a composition in a method of medical treatment, a method comprising administration of such a composition to a patient, e.g. for treatment (which may include preventative treatment) of a medical condition as described above, use of such an agent compound or substance in the manufacture of a composition, medicament or drug for administration for any such purpose, e.g. for treatment of a condition as described herein, and a method of making a pharmaceutical composition comprising admixing such an agent, compound or substance with a pharmaceutically acceptable excipient, vehicle or carrier, and optionally other ingredients. Typically such agents are useful as anti-microbial agents, for example for use as antibiotics to treat bacterial infection in an individual.

In one embodiment the method for providing a pharmaceutical composition may typically comprise:
(a) identifying an agent by an assay method of the invention; and
(b) formulating the agent thus identified with a pharmaceutically acceptable excipient.

The pharmaceutical compositions of the invention may comprise an agent, polypeptide, polynucleotide, vector or antibody according to the invention and a pharmaceutically acceptable excipient.

Whatever the agent used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

An agent or composition may be administered alone or in combination with other treatments, either simultaneously or sequentially dependent upon the condition to be treated, e.g. as described above.
Pharmaceutical compositions according to the present invention, and for use in accordance with the present invention, may include, in addition to active ingredient, a pharmaceutically acceptable excipient, carrier, buffer, stabiliser or other materials well known to those skilled in the art. In particular they may include a pharmaceutically acceptable excipient. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Liposomes, particularly cationic liposomes, may be used in carrier formulations. Examples of techniques and protocols mentioned above can be found in Remington's Pharmaceutical Sciences, 16th edition, Osol, A. (ed), 1980.

The substance or composition may be administered in a localised manner to a particular site or may be delivered in a manner in which it targets particular cells or tissues, for example using intra-arterial stent based delivery.

Targeting therapies may be used to deliver the active substance more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

All the documents cited herein are incorporated herein by reference.

The following Examples illustrate the invention.

### Examples

Materials & Methods for studies leading to the discovery of YcfD as a protein hydroxylase

### YcfD Purification

Recombinant YcfD was produced in N-terminally hexa-His tagged form in E. coli strain BL21 (DE3) using the pET28a vector. To induce expression, 1 mM isopropyl beta-D-1-thiogalactopyranoside (IPTG) was added to cultures at (O)ₐₓ)=0.6, and growth then continued for 4 h at 37° C. Purification of YcfD was carried out by using nickel-affinity chromatography as described elsewhere (Webby et al. Science, 2009: 325 (5936):90-3).

### GFP-Pulldown Experiments

The YcfD gene was cloned into the plasmid pRSet5D containing the GFP coding sequence (Rothbauer et al. Mol. Cell. Proteomics (2008), 7(2):282-9). GFP only expressing cells were used as a control experiment. Either GFP-YcfD or GFP was expressed in E. coli BL21 (DE3) cells. Cells were grown at 37° C. until an OD₆₀₀ of 0.6 Protein expression was induced by isopropyl beta-D-1-thiogalactopyranoside (IPTG) (0.2 mM). 4 hours post-induction cells were harvested and sonicated in tris buffer (50 mM, pH 7.5). Purification of the N-terminally GFP-tagged YcfD or GFP protein was carried out using the GFP-nanotrap (ChromoTek GmbH, Germany) according to the manufactor's instructions. The beads were resuspended in SDS sample buffer and subjected to SDS-PAGE.

### Protein Analysis by Mass Spectrometry

Proteins were separated by 1D SDS-PAGE and stained by using the Colloidal Blue Staining Kit (Invitrogen). Protein bands were excised and digested with trypsin (Promega) according to published protocols (Batycka et al, Rapid Commun. Mass Spectrom (2006), 20(14):2074-80).

The digested material was subjected to nano-ultra performance liquid chromatography tandem MS analysis (nano-UPLC-MS/MS) using a 75 μm-inner diameter×25 cm C₁₈ nanoAcquity™ UPLC™ column (1.7-μm particle size; Waters) and a 90 min gradient of 2-45% solvent B (solvent A: 99.9% H₂O, 0.1% HCOOH acid; solvent B: 99.9% MeCN, 0.1% HCOOH acid) on a Waters nanoAcquity UPLC system (final flow rate, 250 nl/min; 7000 p.s.i.) coupled to a Q-TOF Premier tandem mass spectrometer (Waters) run in positive ion mode. MS analysis was performed in data-directed analysis (DDA) mode (MS to MS/MS switching at precursor ion counts greater than 10 and MS/MS collision energy dependent on precursor ion mass and charge state). All raw MS data were processed using the PLGS software (version 2.3) including deisotoping and deconvolution (converting masses with multiple charge states to m/z=1). The mass accuracy of the raw data was corrected using Glu-fibrinopeptide (200 fmol/μl; 700 nl/min flow rate; 785.8426 Da [M+2H]²⁺) that was infused into the mass spectrometer as a lock mass during analysis. MS and MS/MS data were calibrated at intervals of 30 s. MS/MS spectra (peak lists) were searched against the UniProtKB/Swiss-Prot database (Version 2010.07.16; 518,415 sequences) database using Mascot version 2.3.01 (Matrix Science) and the following parameters: peptide tolerance, 0.2 Da; ¹³C=1; fragment tolerance, 0.1 Da; missed cleavages, 2; instrument type, ESI-Q-TOF; fixed modification, carbamidomethylation (C); and variable modifications, deamidation (N,Q) and oxidation (M,D,K,N,P,R). Analytical runs were repeated with an inclusion list for identified peptides with the highest ion score if arginine hydroxylation was detected. The interpretation and presentation of MS/MS data were performed according to published guidelines (Taylor and Goodlett, Rapid Commun. Mass Spectrom 2005: 19(23):3420). Assignments of hydroxylation on arginine sites identified by Mascot were verified by manual inspection. Ion chromatograms were extracted using the mass windows of ±0.1 Da.

### NMR Analyses

NMR-analyses used a Bruker AVIII 700 system equipped with an inverse TCI cryoprobe optimised for ¹H observation and running TOPSPIN 2 software. Chemical shifts are reported in ppm relative to D₂O (δ_{H} 4.72); the deuterium signal was used as an internal lock signal and the HDO signal was reduced by presaturation where necessary.

For monitoring the YcfD-catalysed decarboxylation of 2-OG to succinate (in absence of a prime substrate), YcfD (20 μM) was added to the assay mixture (1 mM 2OG, 4 mM ascorbate, and 50 μM (NH₄)₂Fe(SO₄)₂, all prepared in deuterated-Tris buffer (pD 7.5, 50 mM in ²H₂O)) directly prior to transfer to a 2 mm NMR tube. The NMR tube was centrifuged for few seconds using a hand centrifuge. The sample was introduced to the magnet and data acquisition was started after a brief optimization (total time lapse between adding the enzyme and the start of data acquisition was 150 seconds). Each acquisition accumulated 16 transients corresponding to 75 seconds of total acquisition time and providing a single spectrum. The delay time between analyses was 0 seconds. The sample temperature was maintained at 310 K throughout the run.

### E. Coli YcfD Knock Out Strain

The YcfD knock out strain, JW1114, was from the E. coli Strain Collection at National Institute of Genetics in Japan (Baba et al, Mol. Syst. Biol. (2006), 2:2006.0008). Inactivation of YcfD was verified by PCR using primers ATGGAATACCAACTCACTC and TTACCCTTCGAAGAACCAATAC (SEQ ID NOs: 8 and 9) consisting of the 5′ end and 3′ end sequences of YcfD gene.

The gene encoding for the L16 protein was PCR amplified and cloned into the pETDuet-1 expression vector. This construct was used to express the L16 protein in the YcfD knock out strain JW1114. For co-expression of L16 and YcfD in the YcfD knock out strain JW1114 both genes were cloned into pETDuet-1.JW1114 cells harbouring pETDuet-1 was grown at 37° C. to an OD₆₀₀ of 0.6 and the expression of the proteins was by induced adding IPTG to the cells at final concentration of 0.5 mM. The cells were grown at 28° C. for further 3 hours before harvest.

### Enzyme Activity Assays

Hydroxylation Assay by MALDI-TOF MS:

Assay mixtures (final volume 100 μL in 50 mM Tris-HCl, pH 7.5) contained: enzyme (10 μM), substrate (100 μM), 2OG (160 μM), Fe(II) (200 μM) and ascorbate (2 mM). The reaction mixture was incubated at 37° C. for 30 minutes. The reaction mixture was then placed on ice, 10 μl of 1% formic acid was added to quench the reaction, and the mixture was centrifuged to separate any insoluble material that precipitated during quenching. The resultant soluble mixture (1 μl) and α-cyano-4-hydroxycinnamic acid (CHCA) matrix solution (1 μl) (LaserBio Labs) were double spotted onto a 96 well MALDI sample plate, and when dry analysed using MALDI-TOF MS on a Waters Micromass™ MALDI micro MX™ mass spectrometer in negative ion reflectron mode.

[¹⁴C] 2OG Decarboxylation Assay:

YcfD was tested for its ability to stimulate decarboxylation of 1-[¹⁴C]-labeled2OG, as described for other 2OG oxygenases (Hewitson et al, J. Biol. Chem. (2002), 277:26351-26355).

¹⁸O₂ Experiment:

Hydroxylation of RLLPAVSEATIRRL (SEQ ID NO: 10) by YcfD was performed under an atmosphere of ¹⁸O₂ as described (Klose et al, Nat. Rev. Genet. 2006, 7(9):715-27). Products were analysed by MALDI-TOF MS.

### Synthesis and Purification of Peptides

Peptides used in this screen were synthesized using a Multipep peptide synthesis machine (Intavis AG Bioanalytical Instruments, Germany) using Fmoc-protected amino acids on a Tentagel S-RAM resin and DIC/HOBT coupling strategy and deprotected by 2.5% Triisopropylsilane/97.5% TFA for three hours. The peptide KPITEKPLAVRMGKGKGNVE (SEQ ID NO: 11) was synthesized on a CS Bio Co 336× peptide synthesizer using similar methods and purified by reverse phase HPLC.

### Results Demonstrating YcfD is a Protein Hydroxylase

To address the question of whether 2OG oxygenase catalysed post translational modifications occur in prokaryotes we carried out structurally informed bioinformatic analyses to identify candidate enzymes. In E. coli we identified four potential 2OG oxygenases, in addition to the already assigned AlkB, a DNA repair enzyme, and taurine dioxygenase (TauD): csiD (GI: 90111476), YcfD (GI: 90111217), ybiU (GI: 16128789), ybiX (GI: 16130149). We chose to focus on YcfD because bioinformatic analyses suggested it is related to human 2OG oxygenases of known (FIH, JMJD6) or proposed function, i.e. the Myc induced nuclear antigen (Mina53).

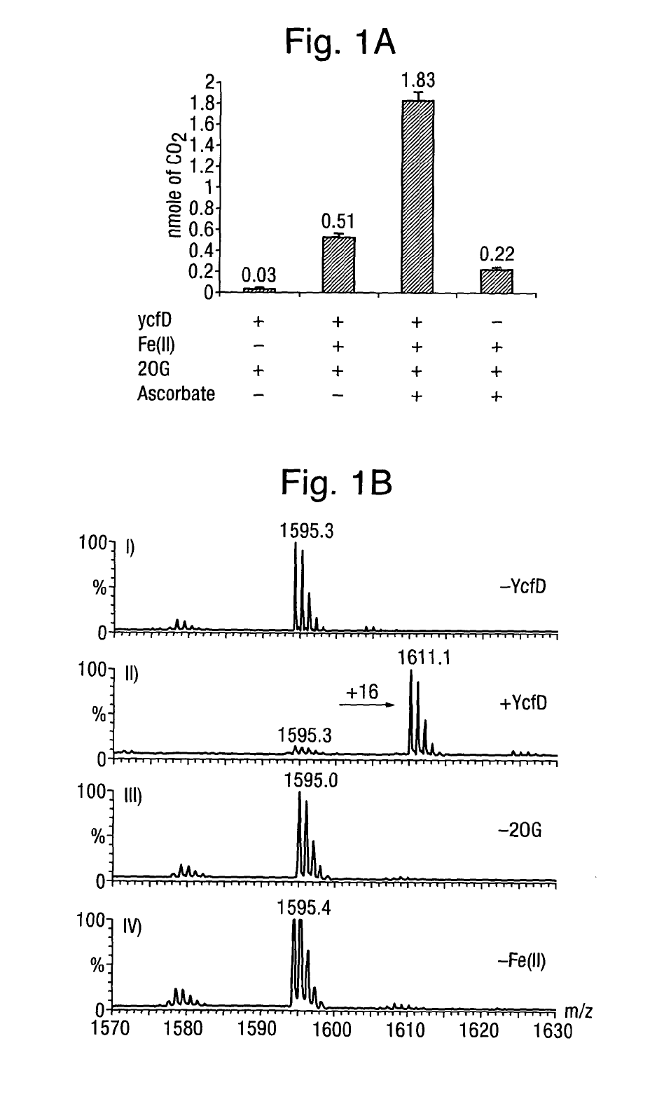

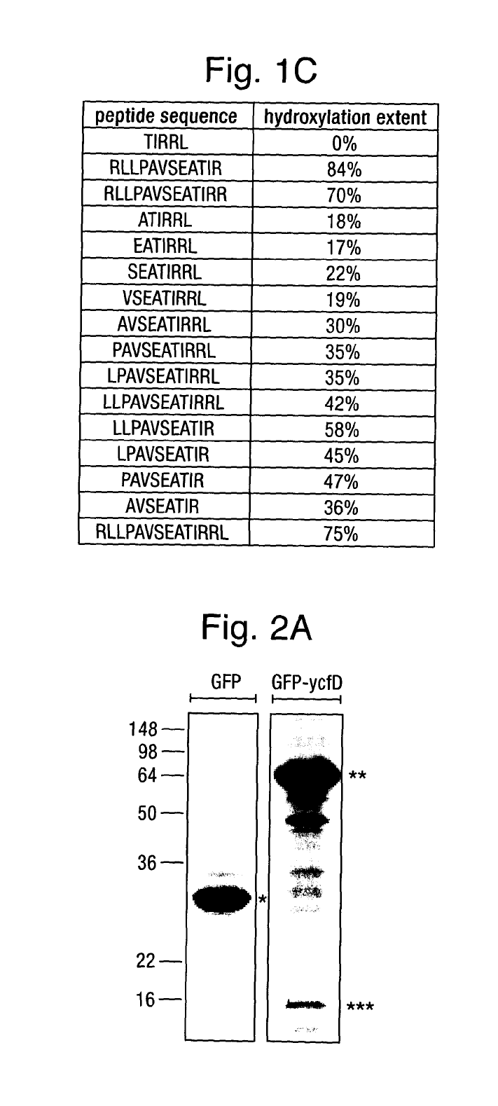

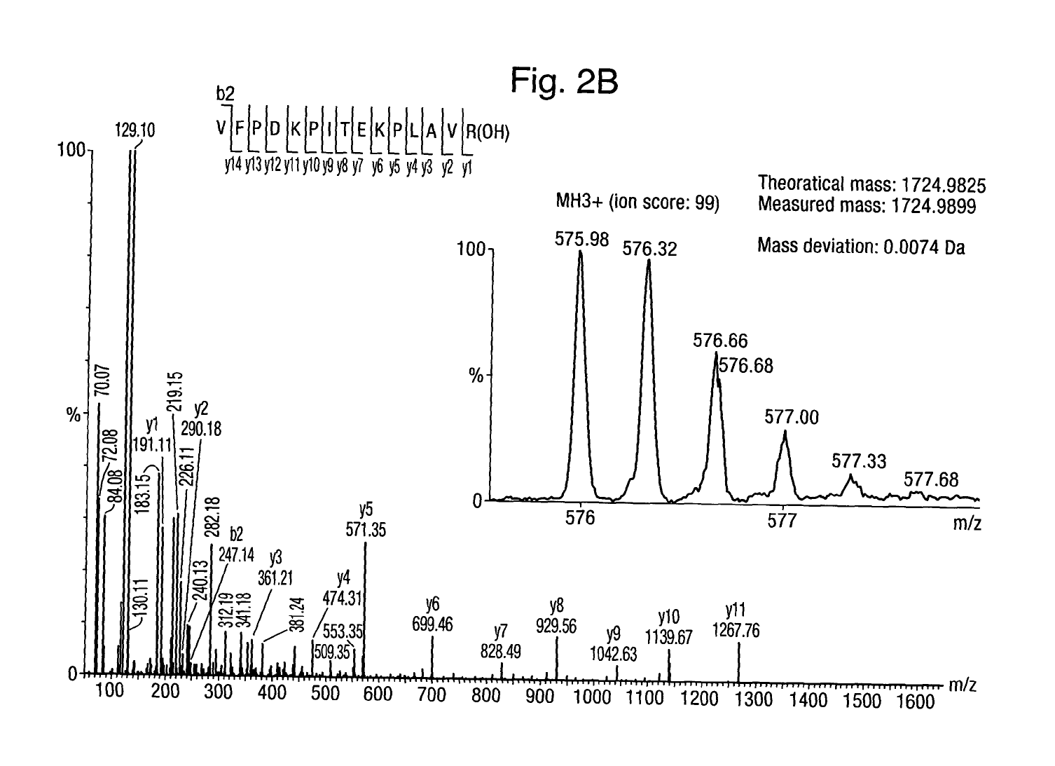

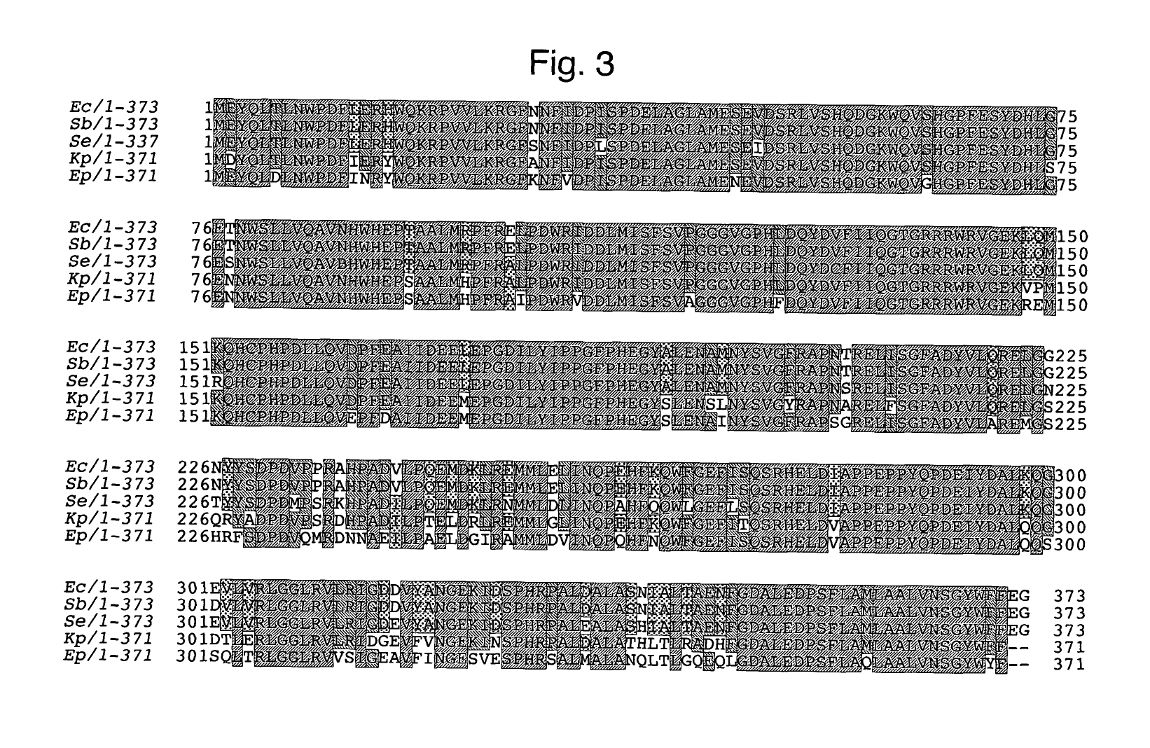

Many, but not all, 2OG oxygenases catalyze substantial turnover of 2OG in the absence of their ‘prime’ substrate. Initially, we therefore prepared purified recombinant YcfD (>90% by SDS-PAGE analysis) and tested it for 2OG turnover activity. We found that YcfD catalyzed 2OG turnover to succinate and CO₂ in an Fe(II) dependent manner; activity was stimulated by the addition of ascorbate and other reducing agents, as observed for many other 2OG oxygenases (FIG. 1A).

Using a mass spectrometric (MS) based assay, we then screened YcfD with 21 known peptide fragments of substrates for three human 2OG dependent hydroxylases (PHD2, FIH, JMJD6), that encompasses known hydroxylation sites, including the HIF-1α N- and C-terminal oxygen dependent degradation domain (prolyl-hydroxylation), the HIF-1α C-terminal transcriptional activation domain (asparaginyl hydroxylation), ankyrin repeat domain peptides (asparaginyl hydroxylation), splicing regulatory protein fragments (lysyl5-hydroxylation), and collagen proyl-hyroxylase peptides. None of these substrates displayed evidence of the +16Da mass shift characteristic of hydroxylation neither did we find that YcfD catalyses Nε-methyl-lysine demethylations of histone H3 fragment peptides, as do some 2OG oxygenases (data not shown). We then screened other available peptides including a set originally prepared for candidate hydroxylases from Pseudomonas spp., that are potential homologues of the human HIF prolyl-hydroxylases. One of these peptides (sequence: RLLPAVSEATIRRL (SEQ ID NO: 10), 14-residues) gave a +16Da mass shift upon incubation with YcfD in an Fe(II) and 2OG dependent manner (FIG. 1B). MS-fragmentation analyses, unexpectedly, implied that the predicted hydroxylation occurred at the arginine-residue (Arg-12) at the -3position relative to the C-terminus of the substrate peptide (data not shown). This proposal was supported by ‘alanine scanning’ analyses hydroxylation was assessed for peptides in which substitution of alanine at every position in turn of RLLPAVSEATIRRL. Hydroxylation was observed in all cases except for substitution at Arg-12. Studies on the variations of the length of peptide required for hydroxylation revealed that of the peptides tested the minimum length required was six residues and also that YcfD can catalyse hydroxylation of C-terminal arginine residues (FIG. 1C). No hydroxylation of L-arginine or N-α-acetyl-L-arginine which are substrates for known prokaryotic 2OG oxygenases or nitric oxide synthase, was observed by ¹H NMR analyses (data not shown). We then employed NMR to investigate the regiochemistry of arginine-hydroxylation, employing an octameric peptide (AVSEATIR) containing a single arginine residue to simplify the analyses. This octameric peptide is set out in SEQ ID NO: 12. Following incubation with YcfD and purification by HPLC, 2D-NMR analyses revealed the position of hydroxylation as at C-3 of the hydroxylated arginine-residue (data not shown). This contrasts with nitric oxide synthase which catalyses hydroxylation of L-arginine to give citrulline and nitric oxide via an Nω-hydroxyarginine intermediate.

Incubation under an ¹⁸O₂ atmosphere confirmed YcfD as a dioxygenase with a high level of incorporation of oxygen from dioxygen (>90%); the high level of ¹⁸O incorporation is consistent with studies on the human 2OG dependent hydroxylases; however, differs from work with 2OG oxygenases with small molecule substrates where, typically, some exchange with oxygen from atmospheric oxygen and water is observed.

Based on the identified peptide substrates for YcfD, we searched the E. coli genome for potential substrates and made a set of peptides. Screening for YcfD activity identified a number of further potential substrate sequences as observed by a +16Da mass shift on products. Examples of the peptide substrates are set out in the Table below.

TABLE 2

| | | % hydroxylation |
|---|---|---|
| Peptide sequence | Protein origin | (+16 Da) |
| LLRLFFPLSLRV (SEQ ID NO: 13) | PhoQ | 50% |
| KLLRLFFPLSLRV (SEQ ID NO: 14) | PhoQ | 60% |
| LAVLQSTLRSLRS (SEQ ID NO: 15) | PhoQ | 60% |
| KTPLAVLQSTLRSLR | PhoQ | 40% |
| (SEQ ID NO: 16) | | |
| LTHSLKTPLAVLQSTLRS | PhoQ | 90% |
| (SEQ ID NO: 17) | | |
| KTPLAVLQSTLRS (SEQ ID NO: 18) | PhoQ | 95% |
| AQYPQEVITTVRG (SEQ ID NO: 19) | PhoP | 60% |
| VEALKERFQASLRD | Phenylalanyl-tRNA synthetase | 40% |
| (SEQ ID NO: 20) | | |
| KHVQALDLSMRFR | Nicotinate-nucleotide | 30% |
| (SEQ ID NO: 21) | diphosphorylase | |
| NTHRGMGYSLRGL | CreB | 30% |
| (SEQ ID NO: 22) | | |
| KWQMMLSKSMRR | CaiF | 30% |
| (SEQ ID NO: 23) | | |
| LTELNREQKWQMMLSKSMRR | CaiF | 40% |
| (SEQ ID NO: 24) | | |
| ELNREQKWQMMLSKSMRR | CaiF | 40% |
| (SEQ ID NO: 25) | | |
| ATVAKCVEALKERFQASLRD | PheT | 40% |
| (SEQ ID NO: 26) | | |
| VAKCVEALKERFQASLRD | PheT | 50% |
| (SEQ ID NO: 27) | | |

Mutation analyses of substrate peptides confirmed arginine as being the preferred residue for hydroxylation in these peptides. In particular, the peptide KTPLAVLQSTLRS (SEQ ID NO: 18) was mutated at arginine with a different amino acid residue in turn, which did not show any hydroxylation other than with the arginine containing peptide. Overall, these results demonstrate that YcfD can catalyse arginine hydroxylation of multiple peptides. Work with human 2OG oxygenases (e.g. Jmjd6) has shown that peptide oxidation is not necessarily a good indication of whether a particular protein is a substrate in cells. Notably, we observed that in some cases when a particular arginine was at the C-terminus of a peptide it was hydroxylated to a greater extend then when in an internal position, suggesting that such peptides may not be representative of in vivo substrates.

In order to identify in vivo relevant YcfD substrates we then carried out co-immunoprecipitation analyses employing green fluorescent protein (GFP)-tagged YcfD and anti-GFP antibody coupled to MS-based identification. One of the identified YcfD binding partners was the ribosomal protein L16 (FIG. 2A). LC-MS/MS analysis of endogenous L16 after immunoprecipitation of GFP-tagged YcfD revealed arginine-81 (R-81) to be completely (>99%) hydroxylated (FIG. 2B). In support of the possibility of YcfD catalysing L16 R-81 hydroxylation, previous proteomic analysis of E. coli ribosomal proteins have also identified R-81 of L16 as being subject to oxidation as observed by a +16Da mass shift.

To test for enzymatic activity of YcfD on R-81 we then made peptide fragments based on the L16 protein sequence and tested them as YcfD substrates in vitro. We found that they were subject to YcfD catalysed hydroxylation in manner dependent on the presence of Fe(II) and 2-oxoglutarate. LC-MS/MS analyses confirmed hydroxylation of R-81 in L16 peptide KPITEKPLAVRMGKGKGNVE (SEQ ID NO: 11) by YcfD protein in vitro (data not shown).

To test whether YcfD catalyses hydroxylation of R-81 in L16 in vivo, we produced His-tagged L16 protein in a YcfD knock out strain (JW1114). MS analyses on the recombinant L16 purified from the YcfD deleted strain demonstrated no detectable levels of arginine hydroxylation on R-81. When YcfD was ectopically expressed in the deletion strain, near complete hydroxylation (with <1% unmodified peptide detected) of R-81 in L16 was observed (data not shown).

Implications of the Finding that YcfD is a Protein Hydroxylase

Our results demonstrate that YcfD is a 2OG oxygenase that catalyses C-3 hydroxylation of R-81 of the E. coli ribosomal protein L16; YcfD is necessary for this post-translational modification. Although various post-translational modifications, including Nω-methylation which is important in transcriptional regulation, to arginine residues have been identified, YcfD is the first enzyme to be found that catalyses post-translational C-3 hydroxylation at arginine-residues. This finding further extends the scope of 2OG oxygenase catalysed oxidations. Importantly, the work demonstrates that 2OG oxygenase catalysed post-translational C-hydroxylation of proteins occurs in prokaryotes. Bioinformatic studies suggest that YcfD homologues are widely distributed and highly conserved in prokaryotes (FIG. 3). Thus, it seems probable that post-translational C-hydroxylation is ubiquitous across all aerobic life forms. Interestingly, 2OG oxygenases also catalyse C-hydroxylation of free arginine/N-acylarginine derivatives during antibiotics biosynthesis in prokaryotes, suggesting an evolutionary link between the amino acid and protein hydroxylases.

In human cells some 2OG dependent hydroxylases have been shown to have multiple protein-substrates. Hence we cannot rule out the possibility that YcfD has also other substrates. Nonetheless, given the roles of 2OG oxygenases in chromatin modification, RNA splicing and in transcriptional regulation by oxygen availability the possibility that translation is regulated by oxygen is important.

The closest human homologues of YcfD are Myc induced nuclear antigen (Mina53) and Nucleolar Protein 66 (NO₆₆). On the basis of bioinformatic analyses and limited cell-based experimental studies, these proteins have been primarily assigned as 2OG dependent Nε-methyl lysine histonedemethylases. Mina53 is highly expressed in some carcinomas and its inhibition suppresses cell proliferation. The assignment of YcfD as an arginine hydroxylase suggests that its human homologs Mina53 and NO66, which has been reported to be a Nε-histonedemethylase may have hydroxylase activity. Reported proteomic results imply that ribosomes undergo a complex matrix of post-translational modifications, that regulate their synthesis and degradation, as well as translation efficiency and accuracy. Notably, given our assignment of YcfD as a ribosomal protein hydroxylase, it is interesting that Mina53 is involved in cell proliferation has been suggested to play a role in ribosome biogenesis.

L16 is an essential component of the bacterial ribosomes, by organizing the conformation of the aminoacyl-tRNA binding site in the 50S subunit, at least in part by interaction with 23S rRNA and 5S rRNA. Biophysical studies on isolated proteins imply R-81 of L16 is located in a relatively disordered loop linking two α-helices in the α+β sandwich fold of L16. Residues in this loop, including R-81, are highly conserved among prokaryotes, but not in archea or eukaryotes (L16 corresponds to L10e in archea). Given the roles of 2OG oxygenase catalysed hydroxylation in collagen and ankyrin fold stabilisation, it is possible that R-81 hydroxylation stabilizes the fold of L16 or its interaction with rRNA or proteins. Mutations in L16 confess resistance to the antibiotic savilamycin and evernimicin in Gram-positive bacteria, including clinically relevant Enterococcus strains. Thus, considerations of whether or not L16, and maybe other ribosomal proteins, are hydroxylated may be relevant in studies on antibiotic action, in particular when they are targeting bacteria in aerobic or hypoxic environments.

1. A method for assaying YcfD activity, the method comprising contacting a peptide containing one or more arginine residues, with a YcfD polypeptide and determining whether an arginine residue in said peptide is hydroxylated.

2. A method according to claim 1 wherein said peptide comprises a ribosomal protein.

3. The method according to claim 1, wherein the peptide is the E. coli ribosomal protein L16.

4. The method according to claim 1 wherein the peptide comprises:
(a) the amino acid sequence of SEQ ID NO: 3
(b) a variant thereof having at least 60% identity to SEQ ID NO: 3 and comprising an arginine equivalent to arginine at position 81 of SEQ ID NO: 3
(c) a fragment of (a) or (b) of at least 6 amino acids in length and comprising arginine at position 81 of SEQ ID NO: 3, or an arginine at a position equivalent to arginine at position 81 of SEQ ID NO: 3.

5. The method according to claim 1, wherein the method is carried out in the presence of Fe(II) and 2-oxoglutarate and optionally in the presence of a reducing agent.

6. The method according to claim 1, wherein the YcfD polypeptide comprises:
(a) the amino acid sequence of SEQ ID NO: 1;
(b) a variant thereof having at least 60% identity thereto and having argininyl hydroxylase activity; or
(c) is a fragment of either thereof having argininyl hydroxylase activity.

7. The method according to claim 1, wherein the assay is carried out in the presence of a test agent to determine whether the test agent is a modulator of YcfD activity, optionally wherein the test agent is a reported inhibitor of a 2-OG oxygenase other than YcfD, or an analogue or variant of such an inhibitor, preferably wherein the inhibitor is an N-oxalyl amino acid such as N-oxalylglycine or a derivative thereof, a glycine or alanine derivative, a 2-oxoacid analogue, a flavonoid or flavonoid derivative such as genistein.

8. A method for:
(a) identifying an inhibitor of YcfD oxygenase activity, the method comprising contacting a YcfD polypeptide and an arginine containing peptide with a test agent under conditions suitable for oxygenase activity, and monitoring for hydroxylation of the arginine of said peptide;
(b) identifying a modulator of protein translation, the method comprising contacting a cell which expresses YcfD with a test agent and determining whether the test agent modulates the YcfD regulation of protein translation;
(c) modulating argininyl hydroxylation by YcfD of a ribosomal protein or a fragment or variant thereof comprising an arginine residue, the method comprising contacting a cell expressing YcfD with an inhibitor or activator of 2-OG oxygenase activity, optionally wherein said cell is in a subject;
(d) modulating protein translation, the method comprising contacting a cell expressing YcfD with an inhibitor or activator of 2-OG oxygenase activity, optionally wherein said cell is in a subject; or
(e) treating bacterial infection, the method comprising administering an inhibitor or activator of 2-OG oxygenase activity to a subject.

9. (canceled)

10. A method according to claim 8, wherein the cell comprises a protein translation reporter construct and the method comprises determining whether YcfD-mediated regulation of protein translation of the reporter construct is modulated by the test agent.

11. The method according to claim 8, wherein the test agent is a reported inhibitor of a 2-OG oxygenase other than YcfD, or an analogue or variant of such an inhibitor.

12. The method of claim 11, wherein the inhibitor is an N-oxalyl amino acid such as N-oxalylglycine or a derivative thereif, a glycine or alanine derivative, a 2-oxoacid analogue, a flavonoid or flavonoid derivative such as genistein.

13-14. (canceled)

15. The method of claim 7, wherein the method further comprises determining whether the test agent modulates the activity of a 2-OG oxygenase other than YcfD (or YcfD homologue), thereby determining whether the test agent selectively modulates the activity of the 2-oxoglutarate dependent oxygenase other than YcfD.

16. A method for introducing hydroxyarginine into a protein by employing a YcfD polypeptide to catalyse such a modification.

17. The method of claim 16 where arginine hydroxylation enables further modification, optionally by glycosylation of the introduced hydroxyl group.

18. A method according to claim 16 such that the hydroxylation alters the properties of the hydroxylated protein.

19. A method according to claim 18 where the hydroxylated protein has increased stability with respect to protease mediated hydrolysis.


SEQUENCE LISTING (27 sequences)

SEQ ID NO: 1 - protein, 373 aa, Escherichia coli
MEYQLTLNWPDFLERHWQKRPVVLKRGFNNFIDPISPDELAGLAMESEVDSRLVSHQDGKWQVSHGPFESYDHLGETNWSLLVQAVNHWHEPTAALMRPFRELPDWRIDDLMISFSVPGGGVGPHLDQYDVFIIQGTGRRRWRVGEKLQMKQHCPHPDLLQVDPFEAIIDEELEPGDILYIPPGFPHEGYALENAMNYSVGFRAPNTRELISGFADYVLQRELGGNYYSDPDVPPRAHPADVLPQEMDKLREMMLELINQPEHFKQWFGEFISQSRHELDIAPPEPPYQPDEIYDALKQGEVLVRLGGLRVLRIGDDVYANGEKIDSPHRPALDALASNIALTAENFGDALEDPSFLAMLAALVNSGYWFFEG

SEQ ID NO: 2 - protein, 128 aa, Escherichia coli
PTAALMRPFRELPDWRIDDLMISFSVPGGGVGPHLDQYDVFIIQGTGRRRWRVGEKLQMKQHCPHPDLLQVDPFEAIIDEELEPGDILYIPPGFPHEGYALENAMNYSVGFRAPNTRELISGFADYVL

SEQ ID NO: 3 - protein, 136 aa, Escherichia coli [sequence omitted]

SEQ ID NO: 4 - protein, 373 aa, Shigella boydii
MEYQLTLNWPDFLERHWQKRPVVLKRGFNNFIDPISPDELAGLAMESEVDSRLVSHQDGKWQVSHGPFESYDHLGETNWSLLVQAVNHWHEPTAALMRPFRELPDWRIDDLMISFSVPGGGVGPHLDQYDVFIIQGTGRRRWRVGEKLQMKQHCPHPDLLQVDPFEAIIDEELEPGDILYIPPGFPHEGYALENAMNYSVGFRAPNTRELISGFADYVLQRELGGNYYSDPDVPPRAHPADVLPQEMDKLREMMLELINQPEHFKQWFGEFISQSRHELDIAPPEPPYQPDEIYDALKQGDVLVRLGGLRVLRIGDDVYANGEKIDSPHRPALDALASNIALTAENFGDALEDPSFLAMLAALVNSGYWFFEG

SEQ ID NO: 5 - protein, 373 aa, Salmonella enterica
MEYQLTLNWPDFLERHWQKRPVVLKRGFSNFIDPLSPDELAGLAMESEIDSRLVSHQDGKWQVSHGPFESYDHLGESNWSLLVQAVNHWHEPTAALMRPFRALPDWRIDDLMISFSVPGGGVGPHLDQYDVFIIQGTGRRRWRVGEKLQMRQHCPHPDLLQVEPFEAIIDEELEPGDILYIPPGFPHEGYALENAMNYSVGFRAPNSRELISGFADYVLQRELGNTYYSDPDMPSRKHPADILPQEMDKLRNMMLDLINQPAHFQQWLGEFLSQSRHELDIAPPEPPYQPDEIYDALKQGEVLVRLGGLRVLRIGDEVYANGEKIDSPHRPALEALASHIALTAENFGDALEDPSFLAMLAALVNSGYWFFEG

SEQ ID NO: 6 - protein, 371 aa, Klebsiella pneumoniae
MDYQLTLNWPDFIERYWQKRPVVLKRGFANFIDPLSPDELAGLAMESEVDSRLVSHQDGKWQVSHGPFESYDHLSENNWSLLVQAVNHWHEPSAALMHPFRALPDWRIDDLMISFSVPGGGVGPHLDQYDVFIIQGTGRRRWRVGEKVPMKQHCPHPDLLQVDPFEAIIDEEMEPGDILYIPPGFPHEGYSLENSLNYSVGYRAPNARELFSGFADYVLQRELGSQRYADPDVPSRDHPADILPTELDRLREMMLGLINQPEHFKQWFGEFITQSRHELDVAPPEPPYQPDEIYDALQQGDTLERLGGLRVLRIDGEVFVNGEKINSPHRPALDALATHLTLRADHFGDALEDPSFLAMLAALVNSGYWFF

SEQ ID NO: 7 - protein, 371 aa, Erwinia pyrifoliae
MEYQLDLNWPDFINRYWQKRPVVLKRGFKNFVDPISPDELAGLAMENEVDSRLVSHQDGKWQVGHGPFESYDHLGENNWSLLVQAVNHWHEPSAALMHPFRAIPDWRVDDLMISFSVAGGGVGPHFDQYDVFIIQGTGRRRWRVGEKREMKQHCPHPDLLQVEPFDAIIDEEMEPGDILYIPPGFPHEGYSLENAINYSVGFRAPSGRELISGFADYVLAREMGSHRFSDPDVQMRDNNAEILPAELDGIRAMMLDVINQPQHFNQWFGEFISQSRHELDVAPPEPPYQPDEIYDALQQSSQLTRLGGLRVVSIGEAVFINGESVESPHRSALMALANQLTLGQEQLGDALEDPSFLAQLAALVNSGYWYF

SEQ ID NO: 8 - DNA, 19 bp, Artificial sequence
  YcfD primer
atggaataccaactcactc

SEQ ID NO: 9 - DNA, 22 bp, Artificial sequence
  YcfD primer
ttacccttcgaagaaccaatac

SEQ ID NO: 10 - protein, 14 aa, Artificial sequence
  Synthetic peptide
RLLPAVSEATIRRL

SEQ ID NO: 11 - protein, 20 aa, Artificial sequence
  Synthetic peptide
KPITEKPLAVRMGKGKGNVE

SEQ ID NO: 12 - protein, 8 aa, Artificial sequence
  Synthetic peptide
AVSEATIR

SEQ ID NO: 13 - protein, 12 aa, Artificial sequence
  Synthetic peptide
LLRLFFPLSLRV

SEQ ID NO: 14 - protein, 13 aa, Artificial sequence
  Synthetic peptide
KLLRLFFPLSLRV

SEQ ID NO: 15 - protein, 13 aa, Artificial sequence
  Synthetic peptide
LAVLQSTLRSLRS

SEQ ID NO: 16 - protein, 15 aa, Artificial sequence
  Synthetic peptide
KTPLAVLQSTLRSLR

SEQ ID NO: 17 - protein, 18 aa, Artificial sequence
  Synthetic peptide
LTHSLKTPLAVLQSTLRS

SEQ ID NO: 18 - protein, 13 aa, Artificial sequence
  Synthetic peptide
KTPLAVLQSTLRS

SEQ ID NO: 19 - protein, 13 aa, Artificial sequence
  Synthetic peptide
AQYPQEVITTVRG

SEQ ID NO: 20 - protein, 14 aa, Artificial sequence
  Synthetic peptide
VEALKERFQASLRD

SEQ ID NO: 21 - protein, 13 aa, Artificial sequence
  Synthetic peptide
KHVQALDLSMRFR

SEQ ID NO: 22 - protein, 13 aa, Artificial sequence
  Synthetic peptide
NTHRGMGYSLRGL

SEQ ID NO: 23 - protein, 12 aa, Artificial sequence
  Synthetic peptide
KWQMMLSKSMRR

SEQ ID NO: 24 - protein, 20 aa, Artificial sequence
  Synthetic peptide
LTELNREQKWQMMLSKSMRR

SEQ ID NO: 25 - protein, 18 aa, Artificial sequence
  Synthetic peptide
ELNREQKWQMMLSKSMRR

SEQ ID NO: 26 - protein, 20 aa, Artificial sequence
  Synthetic peptide
ATVAKCVEALKERFQASLRD

SEQ ID NO: 27 - protein, 18 aa, Artificial sequence
  Synthetic peptide
VAKCVEALKERFQASLRD